(19) United States
(12) Patent Application Publication
(10) Pub. No.: US 2002/0095091 A1
(43) Pub. Date: Jul. 18, 2002

(54) Sphygmomanometer having storage structure

(76) Inventors: Tae Young Che, (US); Jong In Jeung, (US); Won Sub Son, (US)

Correspondence Address: Thomas M. Galgano Esq., Galgano & Burke, 300 Rabro Drive, Suite 135, Hauppauge, NY, 11788, US

(21) Appl. No.: 09/725,584

(22) Filed: Nov. 29, 2000

(30) Foreign Application Priority Data
Jul. 13, 2000 (KR) 2000-20130(U.M.)

(51) Int. Cl. A61B 5/02

(52) U.S. Cl. 600/490

(57) ABSTRACT

Disclosed is a sphygmomanometer comprising: a body having defined therein a receiving space for receiving a blood pressure cuff, the body being formed, on a wall defining the receiving space, with a connector for being connected with the blood pressure cuff through a rubber hose, the body possessing a display section which is disposed above the wall for displaying a measured blood pressure and a control section which is disposed rearward of the wall for calculating a blood pressure of the human body using pulsebeat transferred from the blood pressure cuff; a pair of rotation shafts formed at both side ends of the body, respectively; and a cover capable of being rotated about the pair of rotation shafts for opening and closing the receiving space and the display section of the body.

### BACKGROUND OF THE INVENTION

1. Field of the Invention

The present invention relates to a sphygmomanometer having a storage structure, and more particularly, the present invention relates to a sphygmomanometer having a storage structure which is integrally formed with a receiving structure for receiving a blood pressure cuff thereby eliminating a possibility of the blood pressure cuff to be lost and enabling the blood pressure cuff to be stored in an easy manner and which is additionally provided with a decorative section thereby rendering an aesthetic appeal.

2. Description of the Related Art

Generally, a blood pressure is considered as an important clinical factor due to the fact that the blood pressure is used as a standard for judging a condition of one's health. Consequently, in a hospital, a blood pressure of a patient is fundamentally measured. A blood pressure measuring operation is implemented in a manner such that a blood pressure cuff is wrapped around the upper arm containing the artery, the blood pressure cuff is inflated up to a predetermined pressure thereby to bring the blood pressure cuff into tight contact with the upper arm of the human body and occlude the artery, pulsebeat which is transferred from the artery of the human body is sensed as an electrical signal, and the electrical signal is processed through an electrical circuit to measure a blood pressure of the human body. Recently, as concern of the general public toward health is raised, demand for a portable sphygmomanometer which allows a user to independently and personally measure a blood pressure in an easy manner without going to a hospital, is increased.

An automatic blood pressure measuring device which is a kind of such a portable sphygmomanometer, is disclosed in Korean Patent Publication No. 92-5637 which is published on Jul. 11, 1992. In the automatic blood pressure measuring device, if a user turns on a power switch S1, a motor M is rotated to apply a predetermined air pressure into a blood pressure cuff. If the predetermined air pressure is reached, application of the air pressure into the blood pressure cuff is stopped, and as the air is gradually discharged out of the blood pressure cuff, a blood pressure is measured.

However, the conventional portable sphygmomanometers including the automatic blood pressure measuring device suffer from defects in that, since a separate receiving box is used for storing the blood pressure cuff, a possibility of the blood pressure cuff to be lost is elevated and the sphygmomanometer cannot render any aesthetic appeal.

### SUMMARY OF THE INVENTION

Accordingly, the present invention has been made in an effort to solve the problems occurring in the related art, and an object of the present invention is to provide a sphygmomanometer having a storage structure which is integrally formed with a receiving structure for receiving a blood pressure cuff thereby eliminating a possibility of the blood pressure cuff to be lost and enabling the blood pressure cuff to be stored in an easy manner and which is additionally provided with a decorative section thereby rendering an aesthetic appeal.

In order to achieve the above object, according to one aspect of the present invention, there is provided a sphygmomanometer comprising: a body having defined therein a receiving space for receiving a blood pressure cuff, the body being formed, on a wall defining the receiving space, with a connector for being connected with the blood pressure cuff through a rubber hose, the body possessing a display section which is disposed above the wall for displaying a measured blood pressure and a control section which is disposed rearward of the wall for calculating a blood pressure of the human body using pulsebeat transferred from the blood pressure cuff; a pair of rotation shafts formed at both side ends of the body, respectively; and a cover capable of being rotated about the pair of rotation shafts for opening and closing the receiving space and the display section of the body.

According to another aspect of the present invention, the cover is formed with a decorative section in which a photograph or a picture can be set.

### BRIEF DESCRIPTION OF THE DRAWINGS

The above objects, and other features and advantages of the present invention will become more apparent after a reading of the following detailed description when taken in conjunction with the drawings, in which:

FIG. 1 is a perspective view of a sphygmomanometer having a storage structure in accordance with an embodiment of the present invention;

FIG. 2 is a front view of the sphygmomanometer having a storage structure in accordance with the embodiment of the present invention;

FIG. 3 is a side view of the sphygmomanometer having a storage structure in accordance with the embodiment of the present invention;

FIG. 4 is a side cross-sectional view of the sphygmomanometer having a storage structure in accordance with the embodiment of the present invention;

FIG. 5 is a perspective view illustrating a state wherein the sphygmomanometer having a storage structure in accordance with the embodiment of the present invention is used with a cover opened;

FIG. 6 is a front view illustrating a state wherein the cover of the sphygmomanometer having a storage structure in accordance with the embodiment of the present invention is opened; and

FIG. 7 is a side view illustrating the state wherein the cover of the sphygmomanometer having a storage structure in accordance with the embodiment of the present invention is opened.

### DETAILED DESCRIPTION OF PREFERRED EMBODIMENTS

Reference will now be made in greater detail to a preferred embodiment of the invention, an example of which is illustrated in the accompanying drawings. Wherever possible, the same reference numerals will be used throughout the drawings and the description to refer to the same or like parts.

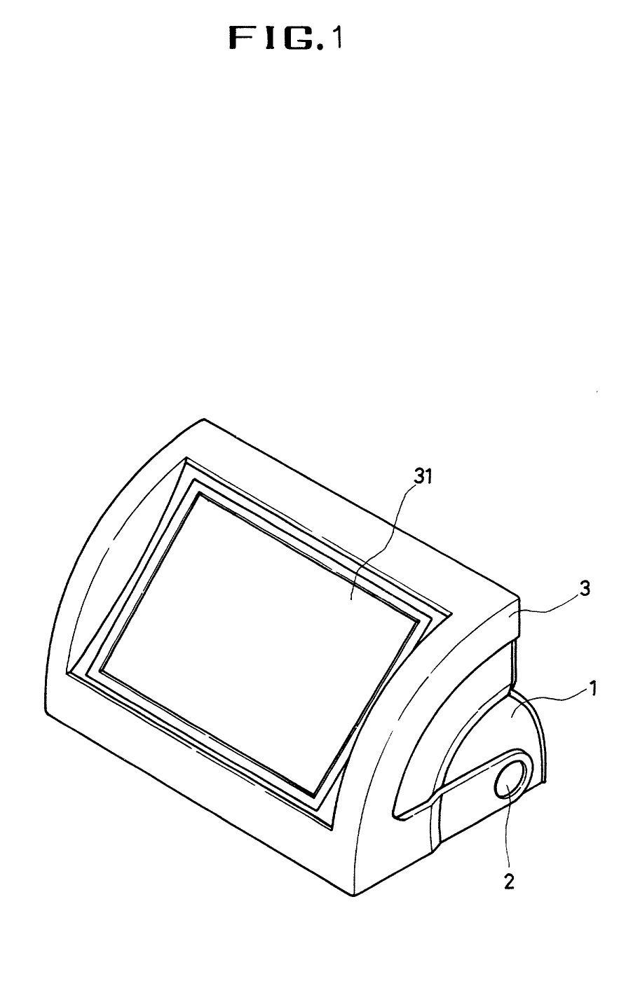

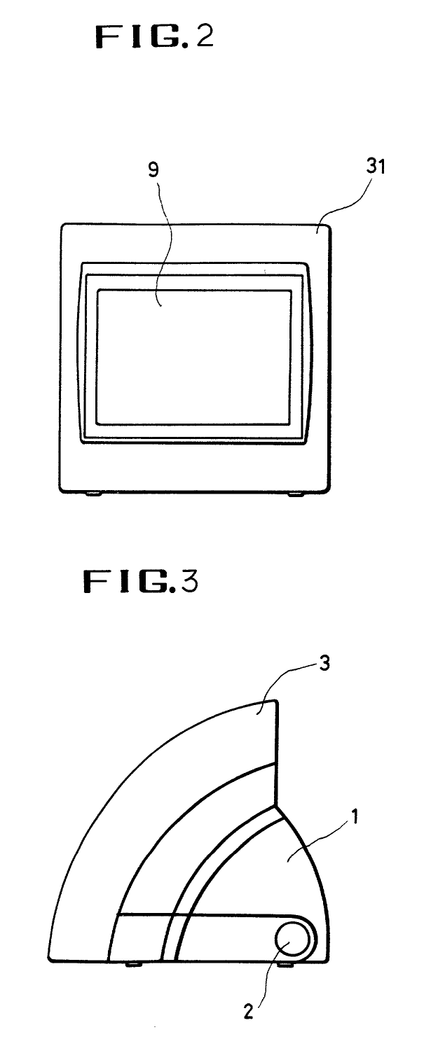

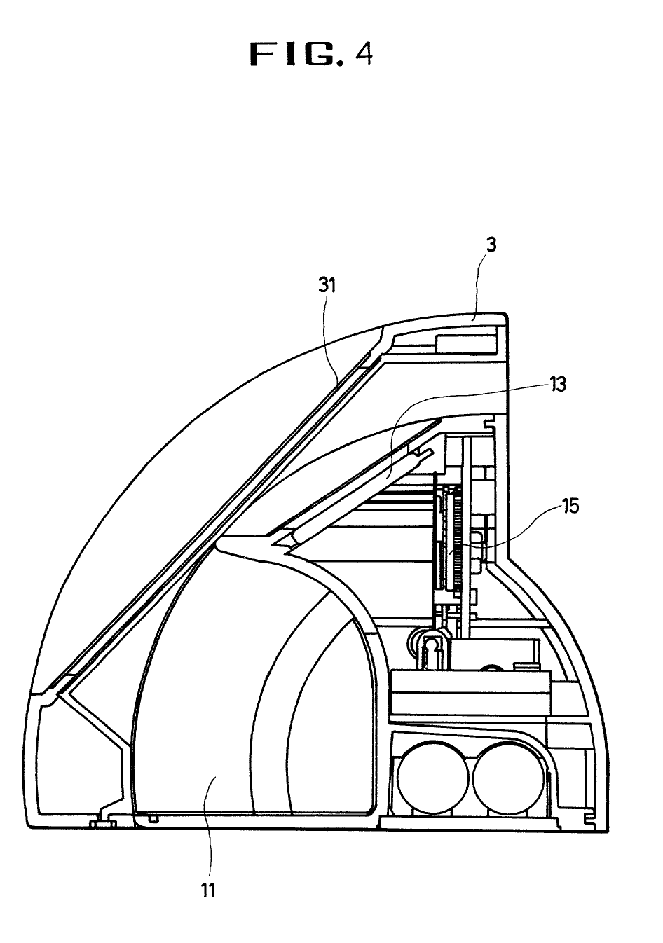

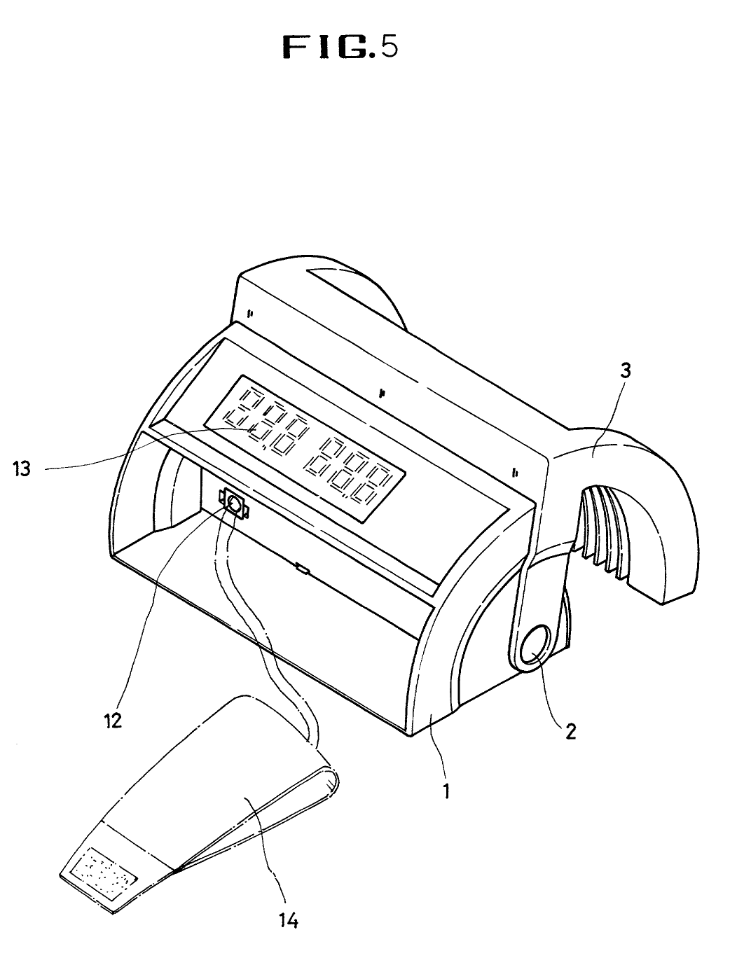

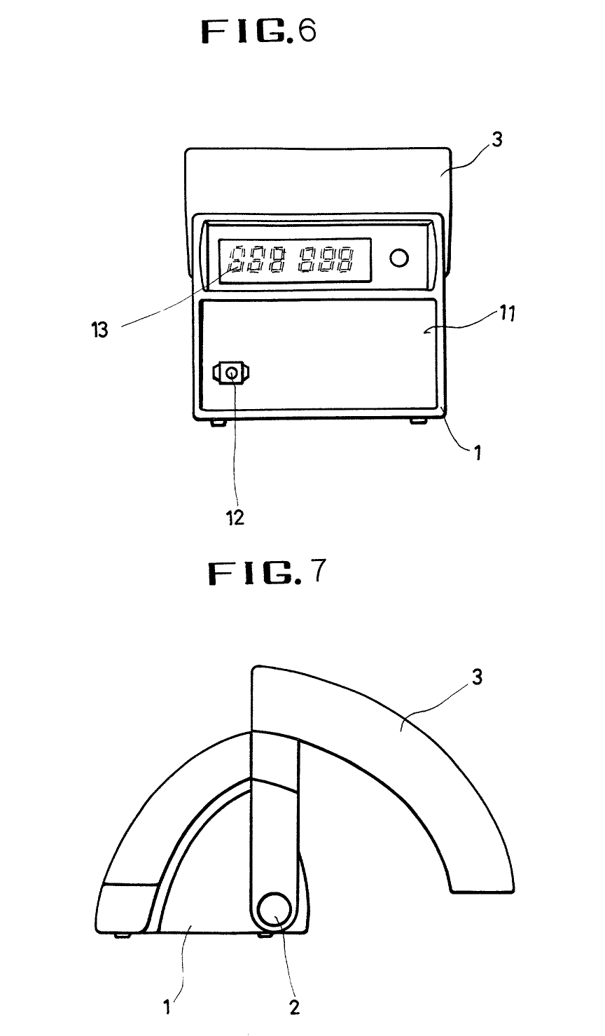

As shown in FIGS. 1 through 7, a sphygmomanometer having a storage structure in accordance with an embodiment of the present invention includes a body 1, a pair of rotation shafts 2 and a cover 3. The body 1 has defined therein a receiving space 11 for receiving a blood pressure cuff 14. The body 1 is formed, on a wall defining the receiving space 11, with a connector 12 for being connected with the blood pressure cuff 14 through a rubber hose. The body 1 possesses a display section 13 which is disposed above the wall for displaying a measured blood pressure and a control section 15 which is disposed rearward of the wall for calculating a blood pressure of the human body using pulsebeat transferred from the blood pressure cuff 14. The pair of rotation shafts 2 are formed at both side ends of the body 1, respectively. The cover 3 can be rotated about the pair of rotation shafts 2 for opening and closing the receiving space 11 and the display section 13 of the body 1. The cover 3 is formed with a decorative section 31 in which a photograph or a picture can be set.

Hereinafter, operations of the sphygmomanometer having a storage structure according to the present invention, constructed as mentioned above, will be described in detail.

If a photograph or a picture is set in the decorative section 31 which is formed in the cover 3, because the decorative section 31 serves as a frame for the photograph or picture, it is possible to enable the cover 3 to be used for a decorative purpose.

When a user desires to measure a blood pressure, the cover 3 is turned over rearward about the pair of rotation shafts 2 in such a way as to open the receiving space 11 of the body 1 and expose the display section 13 of the body 1 to the outside. Then, the blood pressure cuff 14 which is received in the receiving space 11 of the body 1, is taken out, and then, the blood pressure cuff 14 is connected with the connector 12 using the rubber hose. Thereupon, after wrapping the blood pressure cuff 14 around the upper arm of the user, a power switch (not shown) is pressed. Thereafter, as the control section 15 senses fine pulsebeat of the human body, which is transferred from the blood pressure cuff 14, and calculates a blood pressure, the measured blood pressure is displayed on the display section 13 to allow the user to confirm the blood pressure.

If the blood pressure measuring operation is completed, the blood pressure cuff 14 can be disconnected from the connector 12, folded and received in the receiving space 11. Next, by closing the cover 3, the sphygmomanometer can be stored in an easy manner. As described above, by setting a photograph or a picture in the cover 3, the cover 3 can be used for a decorative purpose. By the fact that the photograph or the picture can be changed, it is possible to enjoy a new photograph or picture in obedience to a taste of the user.

As a result, by the sphygmomanometer having a storage structure according to the present invention, advantages are provided in that, since the sphygmomanometer is integrally formed with a receiving structure for receiving a blood pressure cuff, a possibility of the blood pressure cuff to be lost is eliminated and the blood pressure cuff is enabled to be stored in an easy manner. Furthermore, because the sphygmomanometer is additionally provided with a decorative section, an aesthetic appeal is rendered.

In the drawings and specification, there have been disclosed typical preferred embodiments of the invention and, although specific terms are employed, they are used in a generic and descriptive sense only and not for purposes of limitation, the scope of the invention being set forth in the following claims.

What is claimed is:

1. A sphygmomanometer comprising:
a body having defined therein a receiving space for receiving a blood pressure cuff, the body being formed, on a wall defining the receiving space, with a connector for being connected with the blood pressure cuff through a rubber hose, the body possessing a display section which is disposed above the wall for displaying a measured blood pressure and a control section which is disposed rearward of the wall for calculating a blood pressure of the human body using pulsebeat transferred from the blood pressure cuff;
a pair of rotation shafts formed at both side ends of the body, respectively; and
a cover capable of being rotated about the pair of rotation shafts for opening and closing the receiving space and the display section of the body.

2. The sphygmomanometer as claimed in claim 1, wherein the cover is formed with a decorative section in which a photograph or a picture can be set.
